(19) United States
(12) Patent Application Publication
(10) Pub. No.: US 2005/0059582 A1
(43) Pub. Date: Mar. 17, 2005

(54) Use of soluble P-selectin and anthrax lethal toxin

(76) Inventors: Hsin-Hou Chang, Taipei (TW); Der-Shan Sun, Taipei (TW)

Correspondence Address: Ladas & Parry, 26 West 61 Street, New York, NY, 10023, US

(21) Appl. No.: 10/902,489

(22) Filed: Jul. 29, 2004

(30) Foreign Application Priority Data
Jul. 29, 2003 (TW) 092120683

(51) Int. Cl. A61K 38/16

(52) U.S. Cl. 514/8

(57) ABSTRACT

The present invention provides a use of soluble P-selectin in treating systemic hemorrhagic conditions, stabilizing blood pressure, and protecting hypoxic/ischemic tissues. Also provided is a use of anthrax lethal toxin in treating thrombotic conditions.

### FIELD OF THE INVENTION

The present invention relates to novel uses of soluble platelet-selectin (P-selectin) in treating of systemic hemorrhagic conditions, stabilizing blood pressure and protecting hypoxic/ischemic tissues.

### BACKGROUND OF THE INVENTION

Hemorrhagic conditions are diverse in classification, and their pathogenic factors are complicated, including such as abnormality in the structure of vascular walls or connective tissues, abnormality in the amount or function of platelets, abnormality in coagulant factors, over-fibrinolysis, and circulating anti-coagulant substances. Based on the involvement of inheritance, hemorrhagic conditions can be divided into inherited and non-inherited hemorrhagic conditions. Inherited hemorrhagic conditions, such as hemophilia, are resulted from mutations in the coagulant factors, which result in the loss or decrease of coagulation ability, Clinically, most hemorrhagic conditions are non-inherited hemorrhagic conditions, including internal and external traumatic hemorrhage, hemorrhage resulted from insufficient ingestion of nutrition or vitamin K, hemorrhage resulted from gastric or intestinal ulcer, hemorrhage resulted from infectious diseases, hemorrhage resulted from medical behaviors, intrapartum hemorrhage, abnormality in the hematopoietic system, disorders in liver functions, hemorrhage resulted from autoimmune diseases, dengue hemorrhagic fever, hemorrhagic venom attack, anthrax, bacteremia, and hemorrhage resulted from cancer.

Except for traumatic hemorrhage, the present medical treatments have no effect on systemic hemorrhea. The presently known coagulant drugs (such as anti-fibrinolysis agents) are only suitable for treating local hemorrhage. Such coagulant drugs form small thrombus in vivo quickly, and thus often result in side effects such as myocardial infarction and cerebral stroke. Therefore, there exists no suitable and safe systemic haemostatic drugs without side effects.

P-selectin is a member of the selectin family localized in the membranes of α-granules of platelets and the Weibel-Palade bodies (WP bodies) of endothelial cells. Endothelial cells quickly express P-selectin when stimulated by thrombin or histamine. A soluble form of P-selectin (soluble P-selectin; sP-selectin) can be found in the plasma as a circulating protein. A soluble P-selectin molecule, which exists as a monomer in the blood, is 3 kDa smaller than a P-selectin molecule, which exists as an oligomer on a membrane. The soluble P-selectin of healthy individuals originates from the alternatively spliced form found in endothelial cells and platelets (Johnston, G. I. et al., 1990, J. Biol. Chem. 265, 21381-21385).

A previous study showed that the plasma level of soluble P-selectin can be viewed as a useful tool for anticipating thrombotic consumptive platelet disorders (Smith, A. et al., 1999, Throm. Haemost. 82, 1593-1599). Blann, A. D. and Lip, G. Y. indicated that the biological functions of the soluble P-selectin circulating in the blood are unclear (J. Clin. Endocrinol. Metab. (2000) 85, 1745-1747). A Follow-up study utilizing a test determining the plasma clotting time found that soluble P-selectin injected into mice can promote coagulation (Patrick Andre et al., 2000, PNAS Vol. 97, No. 25, 13835-13840). Said study only described the molecular mechanism and local hemorrhage, and inferred that soluble P-selectin may relate to coagulation disorders. However, said study did not indicate that soluble P-selectin can be used in the treatment of systemic hemorrhagic conditions.

Further studies indicated that the plasma soluble P-selectin level of patients suffering from acute myocardial infarction (AMI) and unstable angina pectoris increases significantly (feral Kayikcioglu et al., Int. J. Cardiol., 2001, 79: 223; Enver Atalar et al., Int. J. Cardiol., 2001, 78: 69). However, said literatures did not suggest anything regarding the treatment of systemic hemorrhagic conditions, either.

In view of the above, no literature disclosed or even suggested the novel use of P-selectin in the treatment of systemic hemorrhagic conditions.

### SUMMARY OF THE INVENTION

The present invention provides a use of soluble P-selectin in the manufacture of a medicament for treating systemic hemorrhagic conditions.

The present invention also provides a use of soluble P-selectin in the manufacture of a medicament for stabilizing blood pressure.

The present invention also provides a use of soluble P-selectin in the manufacture of a medicament for protecting is hypoxic/ischemic tissues.

The present invention also provides a use of anti-P-selectin antibodies and P-selectin ligand-1 (PSGL-1) for antagonizing P-selectin.

The present invention also provides a use of anthrax lethal toxin in the manufacture of a medicament for treating thrombotic conditions.

### BRIEF DESCRIPTION OF THE DRAWING

FIG. 1 shows the data obtained in the plasma clotting test. The bars in the figure represent, from top to bottom: Control Immunoglobulin (IgG); Anthrax Lethal Toxin (LeTx); Recombinant Soluble P-selectin (rP-sel); Recombinant Soluble P-selectin (rP-sel)+Anti-P-selectin Antibody (Anti-P-sel); Anti-dengue Non-structural Protein 1 Antibody (Anti-NS1 Antibody)+P-selectin; and Anti-dengue Non-structural Protein 1 Antibody (Anti-NS1 Antibody).

FIG. 2 shows the data obtained in mouse protection test. The data in FIG. 2A represent the protection on mice treated with hemorrhagic snake venoms by soluble P-toxin. The symbol “—◯—” means snake venom of Crotalus atrox+Recombinant Soluble P-selectin (rP-sel); the symbol “—▾—” means snake venom of Crotalus adamanteus+Recombinant Soluble P-selectin (rP-sel); the symbol “—∇—” means snake venom of Crotalus atrox; and the symbol “—▪—” means snake venom of Crotalus adamanteus. The data in FIG. 2B represent the protection on mice treated with anthrax lethal toxin by soluble P-toxin, the symbol “—●—” means Anthrax Lethal Toxin (LeTx); the symbol “—◯—” means Anthrax Lethal Toxin (LeTx)+Recombinant Soluble P-selectin (rP-sel); the symbol “—▾—” means Anthrax Lethal Toxin (LeTx)+Control Immunoglobulin (IgG); and the symbol “—∇—” means Anthrax Lethal Toxin (LeTx)+Recombinant Soluble P-selectin (rP-sel)+Anti-P-selectin Antibody (Anti-P-sel).

FIG. 3 shows the data of platelet aggregation inhibition of anthrax lethal toxin, The symbol “AA” in the figure means arachidonic acid and “LeTx” means anthrax lethal toxin.

FIG. 4 shows the data obtained in the hemorrhage shock mouse model of ischemia/reperfusion. The symbol “—●—” means Control Immunoglobulin (Ig); the symbol “—◯—” means Anti-P-selectin Antibody (Anti-P-sel); the symbol “—▾—” means Recombinant Soluble P-selectin (rP-sel); and the symbol “—∇—” means rP-sel+Anti-P-sel.

FIG. 5 shows the data obtained in the hemorrhage shock mouse model of ischemia. The bars in the figure represent, from left to right: Bovine Serum Albumin (BSA); Recombinant Soluble P-selectin (rP-sel); Control Immunoglobulin (Ig); P-selectin Ligand-1 (PSGL-1); Anti-P-selectin Antibody (Anti-P-sel); and rP-sel+Anti-P-sel.

### DETAILED DESCRIPTION OF THE INVENTION

Currently, there is no effective drug for systemic hemorrhagic conditions which does not have a risk of thrombosis. The current therapies are mostly supporting treatments. There is no better way to treat systemic hemorrhage. The present invention provides the novel use of soluble P-selectin in the treatment of systemic hemorrhagic conditions. Said soluble P-selectin can effectively treat hemorrhagic conditions, particularly anthrax and dengue hemorrhagic fever, and does not induce the side effect of thrombosis. In addition, the soluble P-selectin of the present invention can also be used in the treatment of hemorrhagic conditions induced by bacteremia.

Novel Use of Soluble P-selectin

The present invention provides a use of soluble P-selectin in the manufacture of a medicament for treating systemic hemorrhagic conditions.

Soluble P-selectin and Pharmaceutical Composition Thereof

According to the present invention, the term “soluble P-selectin” comprises naturally occurring soluble forms of P-selectin and recombinant forms thereof, or polymorphic or allelic variants or other isoforms thereof. The term also comprises modified or unmodified soluble P-selectin, such as glycosylated or non-glycosylated forms. Previous studies reveal that soluble P-selectin may originate from activated platelets or endothelial cells. However, the functions of soluble P-selectin and its relationship with platelets are not very clear to persons of ordinary skills in the art. Unexpectedly, the present inventors found that soluble P-selectin provides an excellent effect on the treatment of systemic hemorrhagic conditions. According to the present invention, any forms of soluble P-selectin suitable for treating systemic hemorrhagic conditions can be used in the present invention. Preferably, the soluble P-selectin of the present invention is naturally occurring soluble P-selectin or recombinant soluble P-selectin. According to the present invention, soluble P-selectin can be easily obtained by persons of ordinary skills in the art, such as being isolated from a natural source, purchased from a commercial source, or synthesized with molecular biological techniques.

The soluble P-selectin of the present invention can be prepared into a pharmaceutical composition with a pharmaceutically acceptable carrier. Except for soluble P-selectin and the carrier, said composition may further comprise a diluent, filler, salt, buffer, stabilizing agent, solubilizing agent, and other substances known in the art. The term “pharmaceutically acceptable” refers to non-toxic substances which do not interfere with the biological activity of the active ingredient. The properties of the carrier used in the soluble P-selectin pharmaceutical composition of the present invention are determined based on the administration route. The pharmaceutical composition of the present invention may also comprise a coagulant known in the art, such as vitamin K and anti-fibrinolysis agents.

Administration of Soluble P-selectin

To practice the present invention, a therapeutically effective amount of the soluble P-selectin or pharmaceutical composition of the present invention can be administered to a mammal, including a human or non-human mammal, suffering from hemorrhagic conditions. According to the present invention, the administration of the soluble P-selectin or pharmaceutical composition of the present invention can be carried out by various ordinary ways, such as intravenous administration. When administered in a liquid form, liquid carriers, such as water, saline, and animal or plant oils, may be added. The liquid-form pharmaceutical composition comprises 0.5% to 90% by weight of soluble P-selectin, preferably 1% to 50% by weight of soluble P-selectin.

When a therapeutically effective amount of soluble P-selectin is administered via intravenous injection, the soluble P-selectin is in the form of a parenterally acceptable aqueous solution. The pH, isotonicity, stability and similar conditions to be considered in the preparation of such parenterally acceptable aqueous solution are technology and knowledge known in the art. Except for soluble P-selectin, a preferable pharmaceutical composition for intravenous injection may comprise an isotonic solvent known in the art. The pharmaceutical composition of the present invention may also comprise a stabilizing agent, preservative, buffer, anti-oxidant or other additives known to persons of ordinary skills in the art. The time of intravenous injection treatment with the pharmaceutical composition of the present invention is determined based on the severity of the disease to be treated and the conditions of individual patients. A doctor shall determine the adequate amount of time of intravenous injection treatment with the pharmaceutical composition of the present invention.

The amount of soluble P-selectin in a pharmaceutical composition according to the present invention is determined based on the properties and severity of the disease to be treated and the properties of the treatment previously applied to the patient. A doctor shall determine the amount of soluble P-selectin based on the conditions of individual patients, Regarding the various pharmaceutical compositions for practicing the present invention, it is expected that about 0.1 ng to about 100 mg of soluble P-selectin should be administered per kilogram of bodyweight.

Utility

According to the present invention, soluble P-selectin provides a novel use in the treatment of systemic hemorrhagic conditions. The present inventors unexpectedly found that soluble P-selectin significantly promotes coagulation, but does not induce thrombosis, which results in myocardial infarction and cerebral stroke. Therefore, the soluble P-selectin of the present invention is effective in the treatment of systemic hemorrhagic conditions, and does not have undesirable side effects.

In particular, the soluble P-selectin of the present invention is useful in the treatment of dengue hemorrhagic fever, hemorrhagic snake venoms, anthrax and hemorrhagic conditions in bacteremia. Preferably, the soluble P-selectin of the present invention is useful in the treatment of dengue hemorrhagic fever, hemorrhagic snake venoms and anthrax. Dengue hemorrhagic fever is a systemic hemorrhagic condition resulted from multiple infection of dengue viruses. At the first time of dengue virus infection, most patients only exhibit the symptoms of common colds, and their lives are not threatened. However, after multiple infection of dengue viruses, a specific ratio of patients exhibit life-threatening systemic hemorrhagic conditions. Dengue non-structural protein 1 (NS1) can induce autoimmune antibodies of the host, and the valence of the antibodies increases due to multiple dengue virus infection. Such autoimmune antibodies slow the coagulation rate and result in other possible vascular disorders. Hemorrhagic viper bites are common outdoor risks in Taiwan and other tropical regions. The only effective treatment is the injection of anti-venom serum. However, the species of vipers are numerous. To treat venom poison, specific anti-venom serum must be prepared for each species of vipers. This is a burden which cannot be easily achieved in remote areas. Anthrax, on the other hand, is a disease induced by Becillus anthracis infection. The anthrax lethal toxin produced by Becillus anthracis is the major virulence factor. It has been found that most of the patients died from anthrax exhibit severe internal hemorrhage. The present inventors surprisingly found that soluble P-selectin has a good effect on the treatment of dengue hemorrhagic fever, hemorrhagic snake venoms and anthrax, and does not have any side effect.

In addition, the present inventors also found that soluble P-selectin is very effective in stabilizing blood pressure and protecting hypoxic/ischemic tissues, and is helpful in alleviating relevant symptoms and reducing the death rate. Use of Anti-P-selectin Antibodies and P-selectin Ligand-1 (PSGL-1)

Anti-P-selectin antibodies and P-selectin ligand-1 (PSGL-1) can be used to antagonize the activity of P-selectin, and therefore may be used in the manufacture of medicaments for inhibiting thrombosis and coagulation or modulating oxygen metabolism.

Novel Use of Anthrax Lethal Toxin

The present invention also provides a use of anthrax lethal toxin in the manufacture of a medicament for treating thrombotic conditions. Thrombotic conditions can result in complications such as myocardial infarction and cerebral stroke. Such cardiovascular diseases resulted from thrombosis almost rank among the top ten causes of death. Frequently-used anti-thrombotic drugs include platelet inhibitors, tissue plasminogen activator (tPA), urokinase-type plasminogen activator (uPA), and streptokinase (SK). However, there is still lack of very effective anti-thrombotic drugs. The present inventors surprisingly found that anthrax lethal toxin can effectively inhibit platelet aggregation, and thereby can be used as an anti-thrombotic drug.

According to the present invention, the term “anthrax lethal toxin” comprises naturally occurring anthrax lethal toxin and recombinant forms thereof, or polymorphic or allelic variants or other isoforms thereof. According to the present invention, any forms of anthrax lethal toxin suitable for treating thrombotic conditions can be used in the present invention. Preferably, the anthrax lethal toxin of the present invention is naturally occurring anthrax lethal toxin or recombinant anthrax lethal toxin. According to the present invention, anthrax lethal toxin can be easily obtained by persons of ordinary skills in the art, such as by isolating it from a natural source, purchased from a commercial source, or synthesizing it with molecular biological techniques.

According to the present invention, a therapeutically effective amount of the anthrax lethal toxin or pharmaceutical composition of the present invention can be administered to a mammal, including a human or non-human mammal, suffering from thrombotic conditions. According to the present invention, the administration of the anthrax lethal toxin or pharmaceutical composition of the present invention can be carried out in various ordinary ways, such as oral administration or intravenous injection, preferably intravenous administration. Preferably, it is expected that about 0.01 pg to 500 μg of the anthrax lethal toxin of the present invention should be administered for per kilogram of bodyweight.

The anthrax lethal toxin of the present invention can significantly inhibit platelet aggregation and reduce the risk of complications such as myocardial infarction and cerebral stroke.

### EXAMPLE 1

### Plasma Clotting Test of Dengue Hemorrhagic Fever and Anthrax Lethal Toxin

In the plasma clotting test, the blood from C57BL/6J mice was centrifuged under 1,500×g for 25 minutes to obtain the “platelet-poor plasma” (PPP) of the mice. Equal volume of 20 mM CaCl₂ was added to the obtained plasma, and the change in absorbance resulted from coagulation was recorded under stirring (BOO rpm) and 37° C. on an aggregometer (Model 600B, Ion-Trace, Stouffville, Canada), and the plasma clotting time was determined.

The following reagents were used in the plasma clotting test of this example: recombinant P-selectin-Ig Fc protein (purchased from R & D Systems Inc., Minneapolis, Minn., USA), as the source of P-selectin; dengue non-structural protein 1, as the dengue virus protein, which was obtained from the PCR-synthesized structural protein 1 gene of the Taiwan local strain PLO46 of dengue virus type II (Chang et al., 2002, J. Infect. Dis. 186, 743-51); recombinant anthrax lethal toxin, as the anthrax lethal toxin, which is obtained from a DNA synthesized by the artificial gene synthesizing technique in combination with PCR technique, based on the DNA and protein information (AF065404 and NC-001496) obtained from NCBI Gene Bank, USA (Chang et al., 1993, Biochem. Biophys. Res. Commun. 190, 242-9).

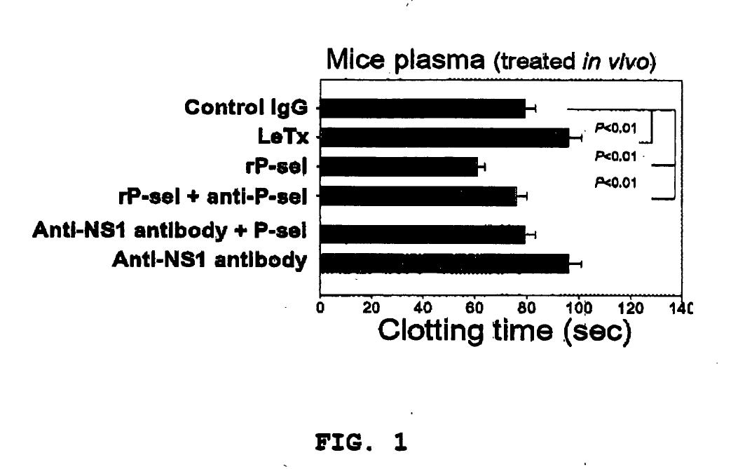

The infusion of the anti-dengue non-structure protein 1 antibody (Anti-NS1 Antibody) and anthrax lethal toxin (LeTx) into the mice inhibited plasma clotting. As shown in FIG. 1, after 24-hour intravenous infusion of Anti-NS1 Antibody and LeTx, the plasma clotting time increased significantly, which implies that the clotting rate decreased. On the contrary, after 24-hour intravenous infusion of recombinant soluble P-selectin (rP-sel), the plasma clotting time decreased significantly, which implies that the clotting rate increased. However, if an anti-P-selectin antibody (Anti-P-sel) was added, the above effect of rP-sel was neutralized. In addition, after 24-hour intravenous infusion of Anti-NS1 Antibody together with soluble P-selectin, the plasma clotting time decreased significantly when compared to the infusion of Anti-NS1 Antibody.

As can be seen from the above results shown in FIG. 1, soluble P-selectin can promote the activation of in vivo coagulation mechanisms. In addition, the infusion of a therapeutic amount of soluble P-selectin alone does not harm the tested animals, but largely increases the coagulation efficiency. Therefore, soluble P-selectin is a very potent candidate of systemic styptic drug.

### EXAMPLE 2

### Mouse Protection Test of Anthrax Lethal Toxin and Hemorrhagic Venoms

In the test of mouse protection by P-selectin, mice were first injected with the recombinant P-selectin-Ig protein, P-selectin+neutralizing monoclonal antibody, or control human immunoglobulin IgG (the dosages are all 1.2 μg/g). After 4 hours, the mice were intravenously injected with anthrax lethal toxin and hemorrhagic venoms. The time of death of the mice were observed. The sources of the recombinant P-selectin-Ig protein and anthrax lethal toxin are shown in Example 1. Venoms of Crotalus atrox and Crotalus adamanteus (purchased from Sigma, St. Louis, Mo., USA) were used as the hemorrhagic venoms.

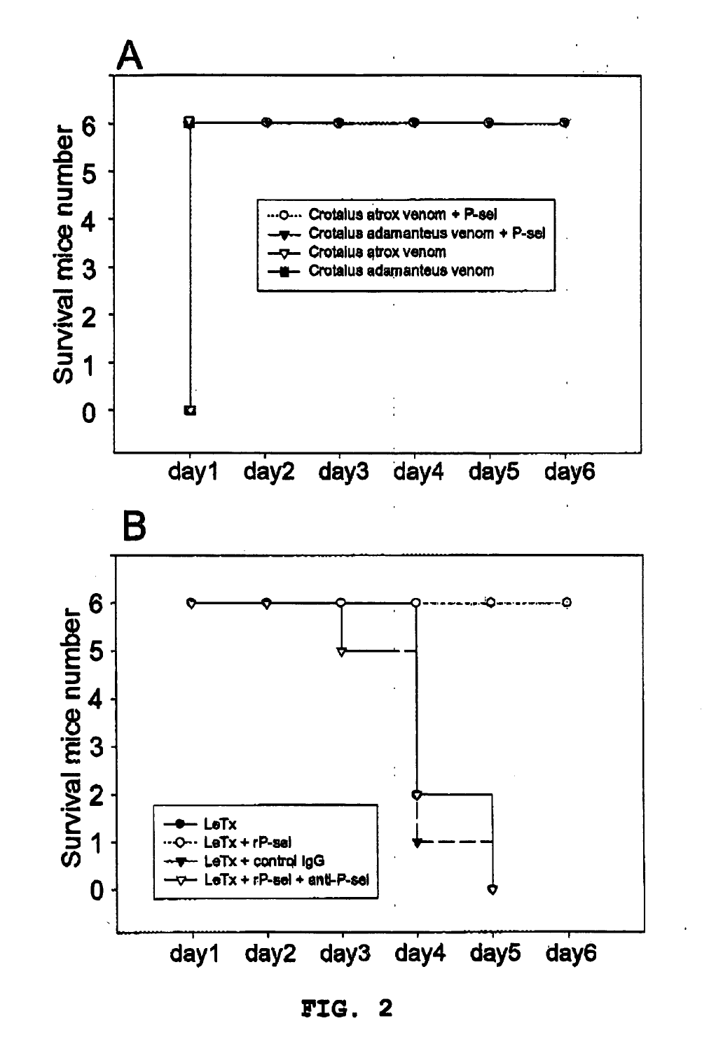

As shown in FIG. 2A, the injected venoms of Crotalus atrox and Crotalus adamanteus each resulted in the death of all the six mice (C57BL/6J) within 1 hour. However, the mice previously injected with recombinant soluble P-protein all survived for more than 3 months, which proves that soluble P-selectin provides protection against hemorrhage. The above test proves that the poisonous damage of hemorrhagic venoms can be treated with soluble P-selectin to avoid death (FIG. 2A). Therefore, soluble P-selectin may be developed into a single systemic anti-hemorrhagic-venom drug.

In addition, as shown in FIG. 23, the injected anthrax lethal toxin (LeTx) resulted in the death of all the six mice (C57BL/6J) within 4-5 days. However, one of the mice previously injected with recombinant soluble P-protein died on the 8^{th} day, another died on the 18^{th} day, and the remaining four mice survived for more than 3 months. The above data proves that soluble P-selectin provides protection against hemorrhage. Moreover, it can be understood from the figure that the protection provided by soluble P-selectin can be neutralized by P-selectin-specific antibodies. Therefore, the above test shows that soluble P-selectin can reduce the number of deaths resulted from anthrax lethal toxin.

### EXAMPLE 3

### Protection Test of Mice with Bacteremia

The reagents and method of the mouse protection test 5 are as shown in Example 2. The bacteremia of mice was simulated by injecting 1×10⁸ bacteria/g of Escherichia coli (E. coli) into the plasma of mice (C57BL/6J). As shown in Table 1, all the six mice injected with the E. coli solution died within 24 hours. However, the mice previously injected with recombinant soluble lo P-protein survived for two more days in average. Therefore, soluble P-selectin does provide mice with protection against bacteremia.

TABLE 1

| Treatment of Mice | Average Surviving Days |
|---|---|
| Injected with Soluble P-selectin | Over 90 Days |
| Injected with E. coli Solution | 1 Day |
| Injected with E. coli Solution + | 3 Day |
| Soluble P-selectin | |

### EXAMPLE 4

### Platelet Aggregation Inhibition Test of Anthrax Lethal Toxin

The effect of anthrax lethal toxin on inhibiting coagulation was tested in the platelet aggregation test. Recombinant anthrax lethal toxin, which is obtained from a DNA synthesized by the artificial gene synthesizing technique in combination with PCR technique. (Chang et al., 1993, Biochem. Biophys. Res. Commun. 190, 242-9), based on the DNA and protein information (AF065404 and NC_{—}001496) obtained from NCBI Gene Bank, USA, was used as the anthrax lethal toxin.

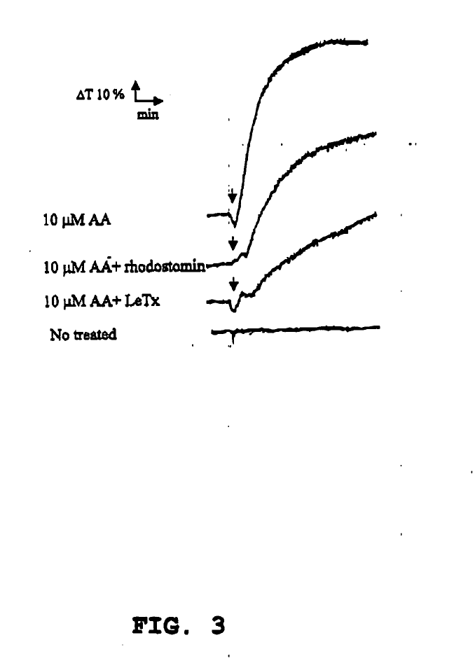

Blood from mice (C57BL/6J) was centrifuged under 500×g for 10 minutes to obtain the “platelet-rich plasma” (PRP) of the mice. The platelet activator, arachidonic acid, was added to the obtained plasma, and the change in absorbance resulted from coagulation was recorded under stirring (800 rpm) and 37° C. on an aggregometer (Model 600B, Ion-Trace, Stouffville, Canada), and the plasma clotting time was determined, As shown in FIG. 3, after treatment with platelet activator, the purified platelets proceeded with normal aggregation. However, the experimental group added with anthrax lethal toxin showed inhibition. Therefore, anthrax lethal toxin can significantly inhibit platelet aggregation.

### EXAMPLE 5

### Hemorrhage Shock Mouse Model of Ischemia/Reperfusion

The effect of soluble P-selectin in stabilizing blood pressure was tested in the hemorrhage shock mouse model of ischemia/reperfusion.

Mice were bled from their vein for about 40% of the total blood volume until their blood pressure lowered to 30-40 mmHg. After maintaining the blood pressure at 30-40 mmHg for 30 minutes, the mice were reperfused with recombinant P-selectin-Ig protein, P-selectin ligand-1 (PSGL-1), neutralizing monoclonal antibody, or control human immunoglobulin IgG (the dosages are all 1.2 μg/g). The changes in blood pressure and survival times of the mice were observed.

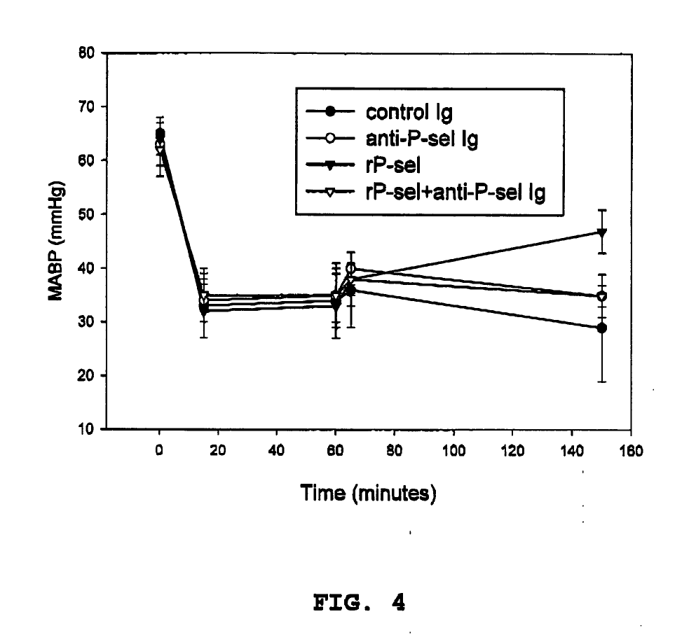

The results shown in FIG. 4 proved that soluble P-selectin can stabilize the low blood pressure resulted from loss of blood in the hemorrhage shock mouse model of ischemia/reperfusion, As shown in FIG. 4, the average blood pressure of the mice treated with soluble P-selectin is higher than that of the mice of the control group. On the other hand, anti-P-selectin antibodies or PSGL-1 can neutralize the blood-pressure-stabilizing effect of soluble P-selectin, and thus can be used as antagonists of soluble P-selectin.

### EXAMPLE 6

### Hemorrhage Shock Mouse Model of Ischemia

The effect of soluble P-selectin in protecting hypoxic/ischemic tissues was tested in the hemorrhage shock mouse model of ischemia.

Mice were intravenously injected with control bovine serum albumin (BSA)O soluble P-selectin, anti-P-selectin antibodies or PSGL-1. After 4 hours, the mice were bled from their vein for about 40% of the total blood volume until their blood pressure lowered to 30-40 mmHg The blood pressure was maintained at 30-40 mmHg for 30 minutes. No reperfusion was given to the mice. The activity of caspase-3 in ischemic tissues was observed for 3 hours.

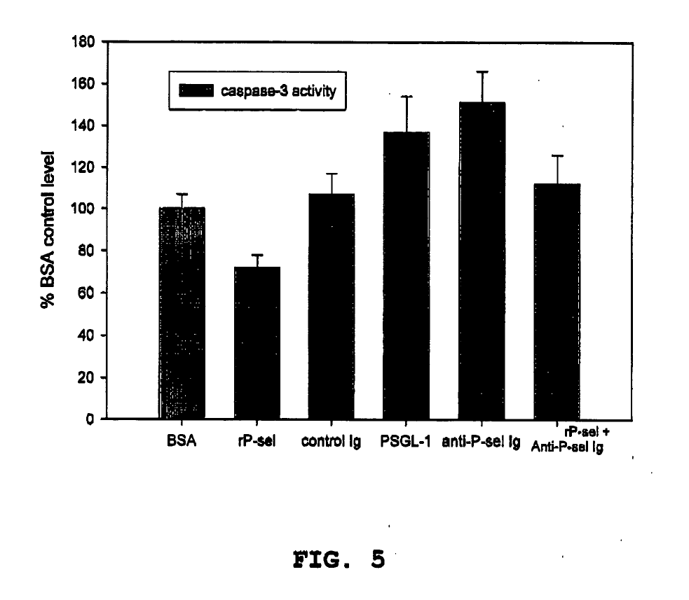

As shown in FIG. 5, the mice pre-treated with soluble P-selectin had the lowest caspase-3 activity, followed by those pre-treated with BSA, and those mice pre-treated with anti-P-selectin antibodies or PSGL-1 had the highest caspase-3 activity, Such results proved that soluble P-selectin can help tissues against a hypoxic environment. On the other hand, anti-P-selectin antibodies or PSGL-1 antagonized the effect of soluble P-selectin.

1. A method of treating systemic hemorrhagic conditions in a mammal, which comprises administering to said mammal a pharmaceutical composition comprising soluble P-selectin and a pharmaceutically acceptable carrier.

2. The method according to claim 1, wherein the soluble P-selectin is selected from the group consisting of naturally occurring soluble P-selectin, recombinant forms thereof, polymorphic variants thereof, allelic variants thereof, and other isoforms thereof.

3. The method according to claim 1, wherein the soluble P-selectin is selected from the group consisting of naturally occurring soluble P-selectin and recombinant forms thereof.

4. The method according to claim 1, wherein the pharmaceutical composition is administered orally or via intravenous injection.

5. The method according to claim 1, wherein soluble P-selectin is administered at an amount of about 0.1 ng to about 100 mg per kilogram of bodyweight.

6. The method according to claim 1, wherein the systemic hemorrhagic condition is selected from the group consisting of dengue hemorrhagic fever, hemorrhagic venoms, and anthrax.

7. The method according to claim 1, wherein the systemic hemorrhagic condition is the hemorrhagic condition resulted from bacteremia.

8. A method of treating thrombotic conditions in a mammal, which comprises administering to said mammal a pharmaceutical composition comprising anthrax lethal toxin and a pharmaceutically acceptable carrier.

9. The method according to claim 8, wherein the anthrax lethal toxin is selected from the group consisting of naturally occurring anthrax lethal toxin, recombinant forms thereof, polymorphic variants thereof, allelic variants thereof, and other isoforms thereof.

10. The method according to claim 8, wherein the anthrax lethal toxin is selected from the group consisting of naturally occurring anthrax lethal toxin and recombinant forms thereof.

11. The method according to claim 8, wherein the pharmaceutical composition is administered orally or via intravenous injection.

12. The method according to claim 8, wherein anthrax lethal toxin is administered at an amount of about 0.01 μg to 500 μg per kilogram of bodyweight.

13. The method according to claim 8, wherein the thrombotic condition is selected from the group consisting of cardiopathy and cerebral stroke.

14. A method of stabilizing blood pressure in a mammal, which comprises administering to said mammal a pharmaceutical composition comprising soluble P-selectin and a pharmaceutically acceptable carrier.

15. The method according to claim 14, wherein the soluble P-selectin is selected from the group consisting of naturally occurring soluble P-selectin, recombinant forms thereof, polymorphic variants thereof, allelic variants thereof, and other isoforms thereof.

16. The method according to claim 14, wherein the soluble P-selectin is selected from the group consisting of naturally occurring soluble P-selectin and recombinant forms thereof.

17. The method according to claim 14, wherein the pharmaceutical composition is administered orally or via intravenous injection.

18. The method according to claim 14, wherein soluble P-selectin is administered at an amount of about 0.1 ng to about 100 mg per kilogram of bodyweight.

19. A method of protecting hypoxic/ischemic tissues in a mammal, which comprises administering to said mammal a pharmaceutical composition comprising soluble P-selectin and a pharmaceutically acceptable carrier.

20. The method according to claim 19, wherein the soluble P-selectin is selected from the group consisting of naturally occurring soluble P-selectin, recombinant forms thereof, polymorphic variants thereof, allelic variants thereof, and other isoforms thereof.

21. The method according to claim 19, wherein the soluble P-selectin is selected from the group consisting of naturally occurring soluble P-selectin and recombinant forms thereof.

22. The method according to claim 19, wherein the pharmaceutical composition is administered orally or via intravenous injection.

23. The method according to claim 19, wherein soluble P-selectin is administered at an amount of about 0.1 ng to about 100 mg per kilogram of bodyweight.
